(19) United States
(12) Patent Application Publication
(10) Pub. No.: US 2024/0180396 A1
(43) Pub. Date: Jun. 6, 2024

(54) MEDICAL IMAGING SYSTEM, MEDICAL IMAGING DEVICE, AND CONTROL METHOD

(71) Applicant: Sony Group Corporation, Tokyo (JP)

(72) Inventor: Yasuaki TAKAHASHI, Tokyo (JP)

(73) Assignee: Sony Group Corporation, Tokyo (JP)

(21) Appl. No.: 18/551,607

(22) PCT Filed: Jan. 25, 2022
(86) PCT No.: PCT/JP2022/002508

§ 371 Date: Sep. 21, 2023

(30) Foreign Application Priority Data
Mar. 31, 2021 (JP) 2021-059288

(51) Int. Cl. A61B 1/00; A61B 1/313; A61B 3/12; G02B 7/36; G06T 7/00

(52) U.S. Cl. CPC A61B 1/000094; A61B 1/00188; A61B 1/3132; A61B 3/12; G02B 7/36; G06T 7/0012; G06T 2207/10068; G06T 2207/10148; G06T 2207/30041

(57) ABSTRACT

A medical imaging system includes a surgery mode setting unit that sets a surgery mode, a region-of-interest setting unit that sets, on the basis of the surgery mode, an ROI image to be used for performing AF processing, from among a Near image focused on a near point, a Mid image focused on a middle point, and a Far image focused on a far point, the Near image, the Mid image, and the Far image being captured by three pieces of imaging elements having different optical path lengths from one imaging lens, and sets a region of interest in the ROI image, and a focus processing unit that obtains an evaluation value from a region of interest of the ROI image, and adjusts focus. The present technology can be applied to, for example, a medical imaging system capable of capturing an EDOF image.

### TECHNICAL FIELD

The present disclosure relates to a medical imaging system, a medical imaging device, and a control method, and more particularly, to a medical imaging system, medical imaging device, and control method that enable display of an appropriately focused image according to surgery.

### BACKGROUND ART

Conventionally, in a medical field, when work such as surgery is performed while an affected area or the like of an image captured through a lens is observed as a region of interest, regions on a front side or back side out of a depth of field are out of focus. For this reason, each time a position of the region of interest shifts to the front side or the back side, focus needs to be adjusted so that the region of interest is in focus, and there is a concern that work efficiency decreases in an image with a shallow depth of field. Therefore, there is a demand for a medical imaging system capable of capturing an image with a deep depth of field.

For example, Patent Document 1 discloses a medical observation device capable of acquiring an extended depth of field (EDOF) image obtained by extending a depth of field.

### CITATION LIST

### Patent Document

Patent Document 1: Japanese Patent Application Laid-Open No. 2017-158764

### SUMMARY OF THE INVENTION

### Problems to be Solved by the Invention

By the way, it is necessary to appropriately control auto focus (AF) depending on a type of surgery so as to obtain an EDOF effect, and, for example, in a case where the EDOF effect cannot be obtained, there may be displayed an image in which, for a near point, a far point in a depth of field is in focus.

The present disclosure has been made in view of such a situation, and an object thereof is to enable display of an appropriately focused image according to surgery.

### Solutions to Problems

A medical imaging system and medical imaging device according to one aspect of the present disclosure include a surgery mode setting unit that sets a surgery mode, a region-of-interest setting unit that sets, on the basis of the surgery mode, an ROI image used for performing AF processing from among two or more types of images captured by at least two pieces of imaging elements having different optical path lengths from one imaging lens, and, in the ROI image, sets a region of interest that is an area for which an evaluation value of contrast AF is obtained, and a focus processing unit that obtains an evaluation value from a region of interest of the ROI image, and adjusts focus.

A control method according to one aspect of the present disclosure includes setting a surgery mode, setting, on the basis of the surgery mode, an ROI image used for performing AF processing from among two or more types of images captured by at least two pieces of imaging elements having different optical path lengths from one imaging lens, and, in the ROI image, setting a region of interest that is an area for which an evaluation value of contrast AF is obtained, and obtaining an evaluation value from a region of interest of the ROI image, and adjusting focus.

In one aspect of the present disclosure, a surgery mode is set, an ROI image used for performing AF processing is set, on the basis of the surgery mode, from among two or more types of images captured by at least two pieces of imaging elements having different optical path lengths from one imaging lens, and, in the ROI image, a region of interest that is an area for which an evaluation value of contrast AF is obtained is set, and an evaluation value is obtained from a region of interest of the ROI image, and focus is adjusted.

### BRIEF DESCRIPTION OF DRAWINGS

FIG. 1 is a diagram illustrating a configuration example of an embodiment of a medical imaging system to which the present technology is applied.

FIG. 2 is a diagram describing a configuration of an endoscope and a device unit.

FIG. 3 is a diagram illustrating a configuration example of an imaging module.

FIG. 4 is a diagram for describing a Mid image, a Near image, a Far image, an EDOF image, and a color-coded image.

FIG. 5 is a diagram illustrating a configuration example of a focus control function of a CCU.

FIG. 6 is a flowchart describing focus control processing.

FIG. 7 is a flowchart describing AF processing.

FIG. 8 is a flowchart describing selection map generation processing.

FIG. 9 is a block diagram illustrating a configuration example of an embodiment of a computer to which the present technology is applied.

### MODE FOR CARRYING OUT THE INVENTION

Hereinafter, a specific embodiment to which the present technology is applied will be described in detail with reference to the drawings.

### Configuration Example of Medical Imaging System

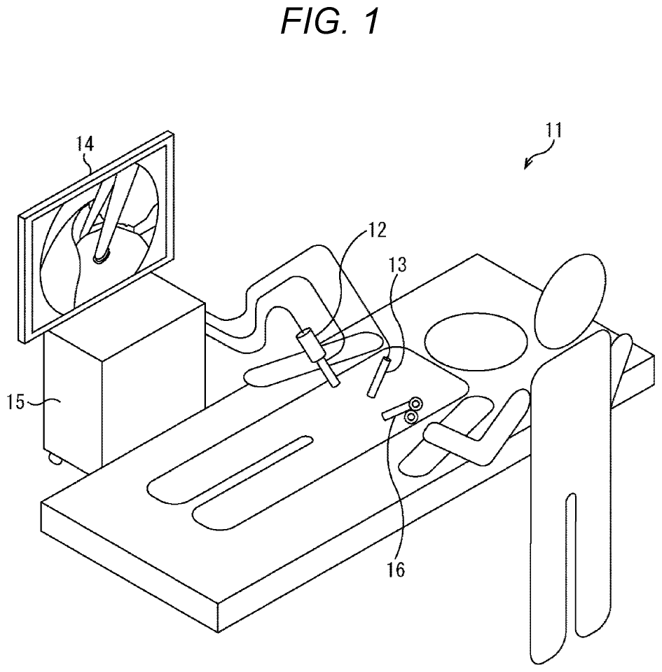

FIG. 1 is a diagram illustrating a configuration example of an embodiment in which a medical imaging system to which the present technology is applied to an endoscopic surgery.

A medical imaging system 11 illustrated in FIG. 1 includes an endoscope 12, an energy treatment tool 13, a display device 14, and a device unit 15.

For example, in a surgery utilizing the medical imaging system 11, the endoscope 12 and the energy treatment tool 13 are inserted into a body of a patient, and forceps 16 are inserted into the body of the patient. Then, in the medical imaging system 11, an image of an affected area such as a tumor imaged by the endoscope 12 is displayed on the display device 14 in real time, and a physician can treat the affected area by using the energy treatment tool 13 and the forceps 16 while viewing the image.

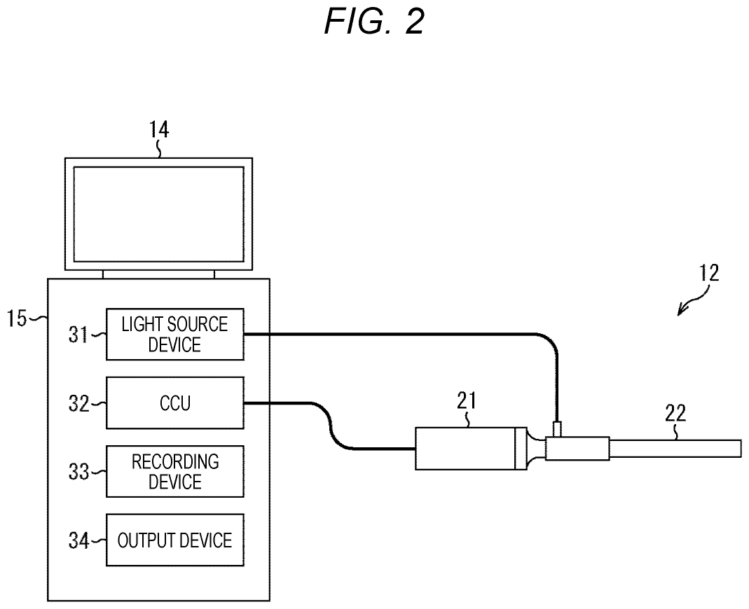

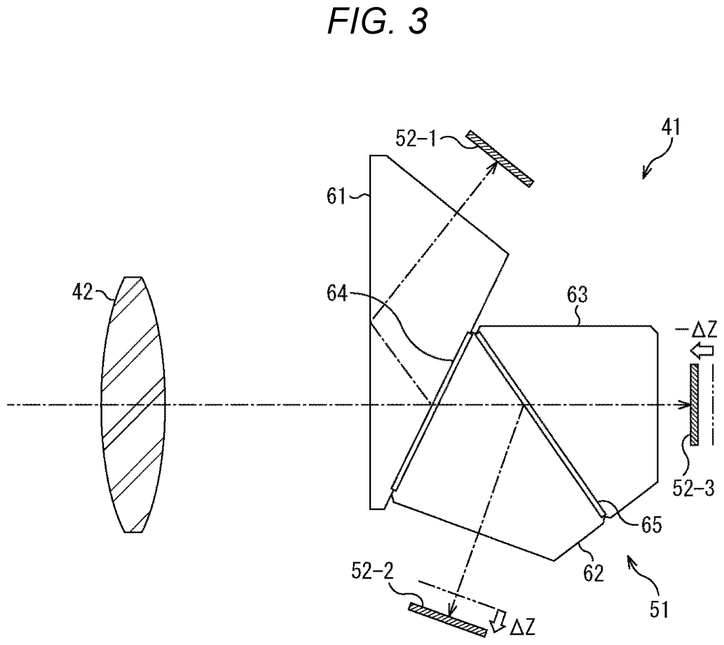

For example, as illustrated in FIG. 2, the endoscope 12 is configured such that a tubular lens barrel unit 22 in which an optical system such as an objective lens is incorporated is mounted on a camera head 21 in which an imaging module (refer to FIG. 3) including a plurality of imaging elements and the like is incorporated. For example, the lens barrel unit 22 is a scope formed into a tubular shape by using a rigid or flexible material, and can guide light to a tip end with a light guide extending inside and emit light on an inside of a body cavity of the patient. The camera head 21 is configured such that an optical element such as, for example, a birefringent mask (BM) can be inserted between the camera head 21 and the lens barrel unit 22, and can capture an image of the inside of the body cavity of the patient via the optical system of the lens barrel unit 22.

The energy treatment tool 13 is, for example, a medical instrument used in an endoscopic surgery, in which an affected area is cut by heat generated by a high-frequency current, or sealing a blood vessel.

The display device 14 can display an image captured by the endoscope 12 as is or can display an image subjected to image processing in the device unit 15.

The device unit 15 is configured by incorporating various devices necessary for performing an endoscopic surgery utilizing the medical imaging system 11. For example, as illustrated in FIG. 2, the device unit 15 can include a light source device 31, a camera control unit (CCU) 32, a recording device 33, and an output device 34.

The light source device 31 supplies, via an optical fiber or the like, the endoscope 12 with light emitted to the affected area when the endoscope 12 performs imaging.

The CCU 32 controls imaging by the endoscope 12 and performs image processing on an image captured by the endoscope 12. Furthermore, the CCU 32 includes, for example, a focus control function for appropriately controlling focus according to a surgery mode when capturing an image with the endoscope 12, an image selection function for appropriately displaying, according to the surgery mode, and the like, an image captured by the endoscope 12.

The recording device 33 records an image output from the CCU 32 on a recording medium. The output device 34 prints and outputs the image output from the CCU 32 or outputs the image via a communication network.

### Configuration Example of Imaging Module

FIG. 3 is a diagram illustrating a configuration example of the imaging module incorporated in the camera head 21 of the endoscope 12.

As illustrated in FIG. 3, an imaging module 41 includes an optical splitting system 51 and three pieces of imaging elements 52-1 to 52-3. Furthermore, an imaging lens 42 is disposed on an optical axis of light incident on the imaging module 41. The imaging lens 42 includes one or a plurality of pieces of lenses, condenses light toward the imaging elements 52-1 to 52-3 such that imaging by light entering the lens barrel unit 22 of the endoscope 12 is performed, and causes the light to enter the optical splitting system 51.

The optical splitting system 51 splits light incident via the imaging lens 42 toward each of the imaging elements 52-1 to 52-3. The optical splitting system 51 includes a first prism 61, a second prism 62, a third prism 63, a first dichroic mirror 64, and a second dichroic mirror 65.

The first prism 61, the second prism 62, and the third prism 63 include prism blocks between the first prism 61 and the second prism 62 and between the second prism 62 and the third prism 63, the prism blocks being joined so as not to generate an air gap. In this manner, by adopting the prism blocks having a so-called gapless structure, it is possible to avoid catching process dust, oozing sealing material, and the like in the optical splitting system 51. Therefore, in the optical splitting system 51, even with a lens system such as, for example, the endoscope 12 having a relatively large F value, it is possible to eliminate reflection of a foreign matter and reduce deterioration in image quality.

The first dichroic mirror 64 is an optical thin film including a dielectric multilayer film formed against an emission surface of the first prism 61, the emission surface being on a side close to the second prism 62, and the first dichroic mirror 64 splits light such that a ratio of, for example, average reflectance : average transmittance=1:2 in light amount.

The second dichroic mirror 65 is an optical thin film including a dielectric multilayer film formed against an emission surface of the second prism 62, the emission surface being on a side close to the third prism 63, and the second dichroic mirror 65 splits light such that a ratio of, for example, average reflectance:average transmittance=1:1 in light amount.

The imaging elements 52-1 to 52-3 are, for example, CMOS image sensors having a Bayer array RGB filter. The imaging element 52-1 is disposed at a position where a distance (optical path length) from a principal point of the imaging lens 42 is an intermediate distance as a reference. The imaging element 52-2 is disposed at a position away from the optical splitting system 51 by a shift amount ΔZ so that the distance from the principal point of the imaging lens 42 is farther than the reference is. The imaging element 52-3 is disposed at a position close to the optical splitting system 51 by the shift amount ΔZ so that the distance from the principal point of the imaging lens 42 is closer than the reference is.

With this arrangement, in a case where a focal length of the imaging lens 42 is adjusted so that the imaging element 52-1 captures an image focused on a region of interest, the imaging element 52-2 captures an image focused on a point closer to a near point than the region of interest is. Similarly, in this case, the imaging element 52-3 captures an image focused on a point closer to a far point than the region of interest is. Therefore, hereinafter, an image captured by the imaging element 52-1 will be referred to as a Mid image, an image captured by the imaging element 52-2 will be referred to as a Near image, and an image captured by the imaging element 52-3 will be referred to as a Far image as appropriate.

Therefore, the imaging module 41 is configured to be able to output the Near image, the Mid image, and the Far image to the CCU 32.

Then, the medical imaging system 11 can switch the Near image, the Mid image, and the Far image and output the switched image to the display device 14, and can switch an EDOF image subjected to image processing in the CCU 32 and a color-coded image and output the switched image to the display device 14.

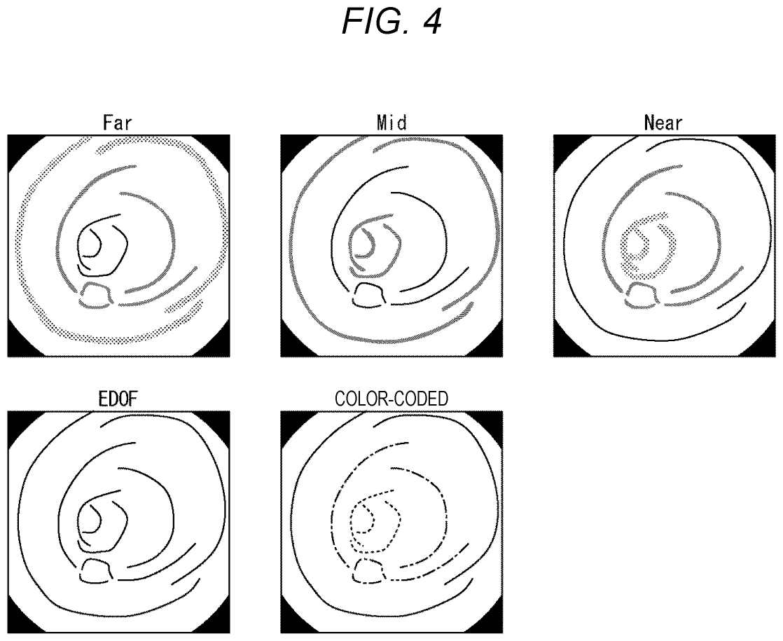

FIG. 4 illustrates images of the Near image, the Mid image, the Far image, the EDOF image, and the color-coded image which are switched and displayed in the medical imaging system 11.

For example, the Near image is captured such that the near point is in focus, in which blur is greater toward a far point side. The Mid image is captured such that a middle point is in focus, in which blur is appearing on the near point side and the far point side. The Far image is captured such that the far point side is in focus, in which blur is greater toward a near point side.

The EDOF image is an image subjected to image processing in which contrast is obtained for each pixel of the Near image, Mid image, and the Far image, and pixels having highest contrast are selected and combined, by which a depth of field is extended so that a range from a near point to a far point is in focus.

The color-coded image is an image obtained by obtaining contrast for each pixel of the Near image, Mid image, and Far image, and color-coding with colors corresponding to the image from which highest contrast is obtained, and the color-coded image is used to select a region. For example, the color-coded image is color-coded such that pixels having highest contrast in the Near image are red (solid lines in FIG. 4), pixels having highest contrast in the Mid image are green (alternate long and short dash lines in FIG. 4), and pixels having highest contrast in the Far image are blue (broken lines in FIG. 4).

Then, in the medical imaging system 11, when a user inputs a surgery mode utilizing, for example, a user interface displayed on the display device 14, the input surgery mode is set in the CCU 32. Then, the CCU 32 can perform focus control so that an appropriately focused image is captured according to the set surgery mode.

For example, in a surgery of an anterior eye, it is desirable to set an ROI image to the Near image, set the region of interest to the cornea appearing in the ROI image, and perform focus control so that the cornea is in focus in the Near image and the crystalline lens is in focus in the Mid image. For example, in a surgery of a fundus region, it is desirable to set an ROI image to the Far image, set the region of interest to the fundus appearing in the ROI image, and perform focus control so that the fundus is in focus in the Far image. In a surgery of a crystalline lens, it is desirable to set the ROI image to the Mid image, set the region of interest to the crystalline lens captured in the ROI image, and perform focus control so that the crystalline lens is in focus in the Mid image. In a laparoscope surgery, it is desirable to set the ROI image to the Mid image, set the region of interest to the center of the ROI image, and perform focus control so that a central portion of the Mid image is in focus.

In this manner, focus control is performed such that the Near image, the Mid image, and the Far image have different focus positions depending on the surgery mode. Moreover, because regions having a highest contrast among the Near image, the Mid image, and the Far image are combined in the EDOF image, it is necessary to set the region of interest focused in order to adjust focus, to respective combined regions.

### Focus Control Function of CCU

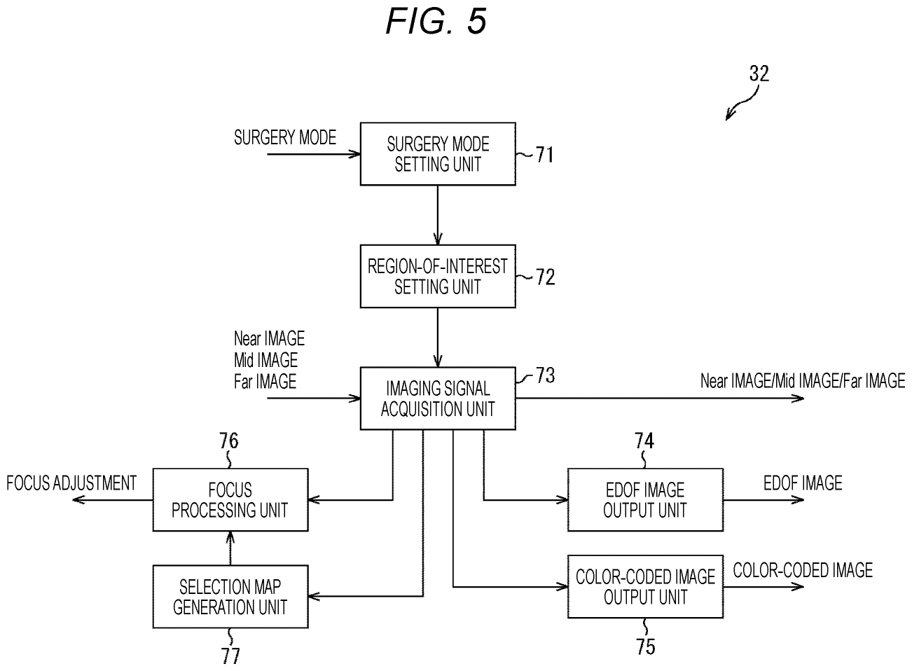

FIG. 5 is a block diagram describing a focus control function of the CCU 32.

As illustrated in FIG. 5, the CCU 32 includes a surgery mode setting unit 71, a region-of-interest setting unit 72, an imaging signal acquisition unit 73, an EDOF image output unit 74, a color-coded image output unit 75, a focus processing unit 76, and a selection map generation unit 77.

For example, when a surgery mode is input by the user by utilizing an input unit (not illustrated), the surgery mode setting unit 71 sets the surgery mode to the region-of-interest setting unit 72.

On the basis of the surgery mode set by the surgery mode setting unit 71, the region-of-interest setting unit 72 sets, to the imaging signal acquisition unit 73, which of the Near image, Mid image, and Far image is to be used as a region of interest (ROI) image to which a region of interest, which is an area for which an evaluation value of contrast AF is to be obtained, is set. Moreover, the region-of-interest setting unit 72 sets a predetermined portion based on the surgery mode as the region of interest in the ROI image.

For example, the region-of-interest setting unit 72 sets the ROI image to the Mid image in a cataract surgery mode, sets the ROI image to the Mid image in a vitreous surgery mode, and sets the ROI image to the Mid image in a laparoscopic surgery mode. Furthermore, the region-of-interest setting unit 72 sets the ROI image to the Near image in a retinal detachment surgery mode, sets the ROI image to the Near image in a corneal transplant surgery mode, and sets the ROI image to the Far image in a macular disease surgery mode. For example, although an image to be used as the ROI image is preset for each surgery mode, it is preferable that the user can change the preset so that an arbitrary image can be set as the ROI image. Note that, in a case where a surgery mode is not set, the ROI image may be set to the Mid image.

Then, in the cataract surgery mode, the region-of-interest setting unit 72 sets, as the region of interest, a portion where an iris is appearing in a central portion of a screen. For example, to which position of an eye medical staff sets an imaging region in an ophthalmic surgery is conventionally decided, and therefore, it is preferable to hold the region of interest for each surgery mode in advance, with a table. Note that a configuration may be adopted in which a detection target and an image recognition algorithm are decided with image recognition on the basis of a surgery mode to detect the region of interest.

The imaging signal acquisition unit 73 acquires imaging signals of the Near image, Mid image, and Far image output from the imaging module 41. Then, from among the Near image, the Mid image, and the Far image, the imaging signal acquisition unit 73 sets an image set by the region-of-interest setting unit 72 as the ROI image, and sets the region of interest to the ROI image. The imaging signal acquisition unit 73 supplies the focus processing unit 76 with the ROI image to which the region of interest is set. The imaging signal acquisition unit 73 supplies the Near image, the Mid image, and the Far image to the EDOF image output unit 74, the color-coded image output unit 75, and the selection map generation unit 77. The imaging signal acquisition unit 73 can switch any one of the Near image, the Mid image, and the Far image and output the switched image to the display device 14.

The EDOF image output unit 74 obtains contrast for each pixel of the Near image, Mid image, and Far image, and selects and combines pixels having highest contrast, thereby outputting an EDOF image with a depth of field extended so that a range from a near point to a far point is in focus.

The color-coded image output unit 75 obtains contrast for each pixel of the Near image, Mid image, and Far image, thereby outputting a color-coded image color-coded with colors corresponding to the image from which highest contrast is obtained. For example, the color-coded image is used to select a region.

The focus processing unit 76 controls an optical system of the endoscope 12 so that the center of the ROI image supplied from the imaging signal acquisition unit 73 is most in focus, and adjusts focus of when imaging is performed by the imaging module 41. Furthermore, the focus processing unit 76 may use wobbling to roughly calculate and align the focus position in the ROI image. Moreover, the focus processing unit 76 performs fine focus adjustment by using a selection map generated by the selection map generation unit 77.

The selection map generation unit 77 generates a selection map used to finely adjust the focus for when imaging is performed by the imaging module 41, and supplies the selection map to the focus processing unit 76.

For example, the selection map generation unit 77 obtains contrast for each pixel of the Near image, Mid image, and Far image, and selects, from among the Near image, the Mid image, and the Far image, an image from which highest contrast is obtained. That is, the selection map generation unit 77 compares contrast with peripheral pixels in the same image, and selects an image having a pixel having a largest difference (highest in contrast). The selection map generation unit 77 associates an identification number for identifying a selected image (number assigned to each of the Near image, Mid image, and Far image) with each individual pixel. Then, the selection map generation unit 77 can generate a selection map by associating the identification number with all the pixels.

Therefore, in the CCU 32, the focus processing unit 76 can finely adjust the focus by using the selection map generated by the selection map generation unit 77.

For example, every time focus adjustment is repeated, the focus processing unit 76 determines whether or not an identification number associated with a pixel of the region of interest in the selection map generated from an image captured with focus adjusted immediately before matches an image set as the ROI image. Then, in a case where the identification number of the region of interest in the selection map does not match the image set as the ROI image, the focus processing unit 76 adjusts the focus so that the identification number of the region of interest in the selection map matches the image set as the ROI image. At this time, the focus processing unit 76 may move the focus lens in a predetermined direction by a predetermined amount, or may decide, on the basis of statistics of contrast of the region of interest in the selection map, in which direction and how much the focus lens moves.

Thereafter, the focus processing unit 76 performs contrast AF on the region of interest of the ROI image when the identification number of the region of interest in the selection map matches the image set as the ROI image. With this arrangement, it is possible to bring the region of interest of the ROI image into focus in the

EDOF image. Note that determination as to whether or not the identification number of the region of interest in the selection map matches the image set as the ROI image may be not only an exact match (100%) but also a match at a predetermined rate (80% or the like), for example.

The focus control function of the CCU 32 is configured as described above, and an optimal EDOF image can be acquired according to the surgery mode by simultaneously acquiring the Near image, the Mid image, and the Far image and changing an autofocus evaluation value calculation method according to the surgery mode. With this arrangement, it is possible to obtain an EDOF effect that achieves optimal depth of field extension when performing a surgery, and as a result, it is possible to improve work efficiency.

Note that the contrast AF is similar to focus evaluation in general AF, and the focus processing unit 76 sets a plurality of focus frames in an image, evaluates a luminance value in the focus frames, and decides movement amount and movement direction of the focus lens by viewing a correlation for each focus frame. Note that the focus lens can be included in the imaging lens 42 in FIG. 3.

Then, the focus processing unit 76 decides the movement amount and movement direction of the focus lens by using focus evaluation of the focus frame overlapping the region of interest. Note that the focus evaluation is always performed in all the focus frames, and an evaluation result of which focus frame is used is selected depending on the surgery mode. Furthermore, in general, in a medical field, only a contrast detection method is used, but phase difference detection may be used together.

Moreover, the CCU 32 may adopt a configuration in which the focus is adjusted after the focus position is changed at a high speed by the user switching the Near image, the Mid image, the Far image, or the EDOF image, for example. That is, it is preferable to switch the ROI image from the Near image to the Mid image as the user switches the image from the Near image to the Mid image. At this time, when the regions of interest of the Near image, Mid image, and Far image are preset depending on the surgery mode, the region of interest is also simultaneously switched. With this arrangement, when the image is switched from the Near image to the Mid image, a focus condition can also be changed, by which more appropriate autofocus can be achieved.

### Processing Example of Focus control processing

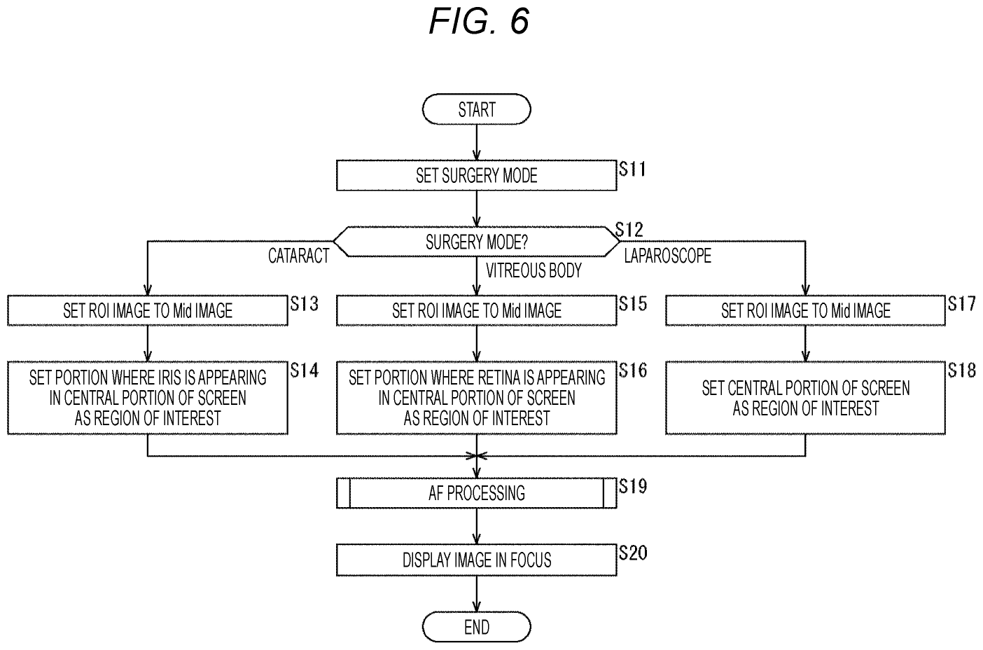

Focus control processing will be described with reference to flowcharts illustrated in FIGS. 6 to 8.

FIG. 6 is a flowchart describing focus control processing executed by the CCU 32. Note that there will be described in FIG. 6 a processing example in which any one of the cataract surgery mode, the vitreous surgery mode, and the laparoscopic surgery mode is set as the surgery mode, but similar processing is performed in a case where various surgery modes as described above are set.

For example, when a surgery mode is input by the user, processing is started, and in Step S11, the surgery mode setting unit 71 sets the surgery mode input by the user to the region-of-interest setting unit 72.

In Step S12, the region-of-interest setting unit 72 determines whether the surgery mode set in Step S11 is the cataract surgery mode, the vitreous surgery mode, or the laparoscopic surgery mode.

In a case where the region-of-interest setting unit 72 determines in Step S12 that the cataract surgery mode has been set, the processing proceeds to Step S13. The region-of-interest setting unit 72 sets in Step S13 the ROI image to the Mid image, and sets in Step S14 the portion where the iris is appearing in the central portion of the screen of the ROI image as the region of interest.

Meanwhile, in a case where the region-of-interest setting unit 72 determines in Step S12 that the vitreous surgery mode has been set, the processing proceeds to Step S15. The region-of-interest setting unit 72 sets in Step S15 the ROI image to the Mid image, and sets in Step S16 the portion where a retina is appearing in the central portion of the screen of the ROI image as the region of interest.

Meanwhile, in a case where the region-of-interest setting unit 72 determines in Step S12 that the laparoscopic surgery mode has been set, the processing proceeds to Step S17. The region-of-interest setting unit 72 sets in Step S17 the ROI image to the Mid image, and sets in Step S18 the central portion of the screen of the ROI image as the region of interest. For example, in a case where there is an affected area such as a tumor to be subjected to a surgery, the endoscope 12 is operated so that the affected area appears in the central portion of the screen of the ROI image, by which the region-of-interest setting unit 72 can set the portion where the affected area is appearing as the region of interest.

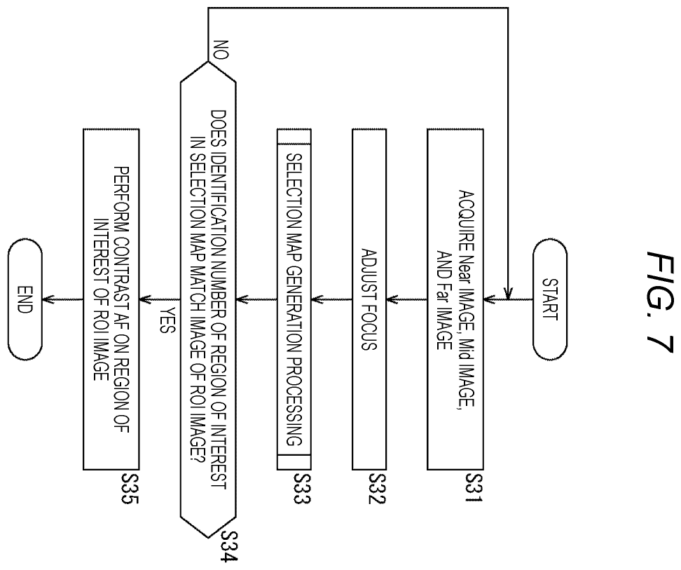

After the processing in Step S14, Step S16, or Step S18, the processing proceeds to Step S19, and AF processing (refer to FIG. 7 described later) is executed in which the focus is adjusted so that the region of interest set as the ROI image according to the surgery mode is in focus.

In Step S20, an image selected from among the Near image, the Mid image, and the Far image according to the surgery mode, the image being adjusted to be in focus by the AF processing in Step S19 and captured, is output from the CCU 32 and displayed on the display device 14. FIG. 7 is a flowchart describing the AF processing executed in Step S19 in FIG. 6.

In Step S31, the imaging signal acquisition unit 73 acquires imaging signals of the Near image, Mid image, and Far image output from the imaging module 41.

In Step S32, the imaging signal acquisition unit 73 supplies the focus processing unit 76 with the ROI image set by the region-of-interest setting unit 72 in Step S13, Step S15, or Step S17 in FIG. 6. Then, the focus processing unit 76 controls the optical system of the endoscope 12 so that the center of the ROI image is most in focus. With this arrangement, the focus is adjusted so that an imaging element 52, among the imaging elements 52-1 to 52-3 of the imaging module 41, that captures the image set as the ROI image is in focus, and the imaging signals of the Near image, Mid image, and Far image captured with the focus are supplied from the imaging signal acquisition unit 73 to the selection map generation unit 77.

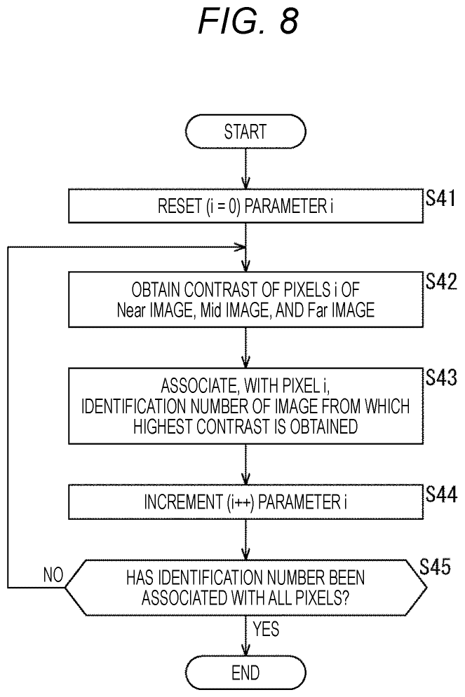

In Step S33, the selection map generation unit 77 executes selection map generation processing (refer to FIG. 8 described later) of generating a selection map by using the imaging signals of the Near image, Mid image, and Far image supplied from the imaging signal acquisition unit 73 in Step S32.

In Step S34, the selection map generation unit 77 determines whether or not the identification number associated with the pixel of the region of interest in the selection map generated in the selection map generation processing in Step S33 matches the image set as the ROI image.

In a case where the selection map generation unit 77 determines in Step S34 that the identification number associated with the pixel of the region of interest of the selection map does not match the image set as the ROI image, the processing returns to Step S31, and similar processing is repeatedly performed thereafter.

Meanwhile, in Step S34, in a case where the selection map generation unit 77 determines that the identification number associated with the pixel of the region of interest of the selection map matches the image set as the ROI image, the processing proceeds to Step S35.

In Step S35, the imaging signal acquisition unit 73 acquires the ROI image captured with a final focus adjusted by repeatedly performing the processing in Steps S31 to S33, and supplies the acquired ROI image to the focus processing unit 76. Then, after the focus processing unit 76 obtains an evaluation value and performs contrast AF so that the region of interest set as the ROI image is in focus, the AF processing ends.

FIG. 8 is a flowchart describing the selection map generation processing executed in Step S33 in FIG. 7. In Step S41, the selection map generation unit 77 resets (i=0) the parameter i that specifies the pixel to be processed.

In Step S42, the selection map generation unit 77 obtains contrast for each pixel i of the Near image, Mid image, and Far image supplied from the imaging signal acquisition unit 73 in Step S32 in FIG. 7.

In Step S43, the selection map generation unit 77 specifies, from among the contrast of the pixel i of the Near image, the contrast of the pixel i of the Mid image, and the contrast of the pixel i of the Far image that are obtained in Step S42, an image from which highest contrast is obtained. Then, the selection map generation unit 77 associates an identification number for identifying the image (Near image, Mid image, or Far image) from which the highest contrast is obtained with the pixel i to be processed.

In Step S44, the selection map generation unit 77 increments (i++) the parameter i that specifies the pixel to be processed.

In Step S45, the selection map generation unit 77 determines whether or not the identification number has been associated with all the pixels of the Near image, Mid image, and Far image supplied from the imaging signal acquisition unit 73 in Step S32 in FIG. 7. For example, in a case where the parameter i matches the number of pixels, the selection map generation unit 77 can determine that identification number has been associated with all the pixels.

In a case where the selection map generation unit 77 determines in Step S45 that the identification number has not been associated with all the pixels of the Near image, Mid image, and Far image, the processing returns to Step S42, and similar processing is repeatedly performed thereafter.

Meanwhile, in a case where the selection map generation unit 77 determines in Step S45 that the identification number has been associated with all the pixels of the Near image, Mid image, and Far image, the processing ends. That is, in this case, a selection map is generated in which an identification number for identifying any one of the Near image, the Mid image, and the Far image is associated with all the pixels.

As described above, in the medical imaging system 11, AF can be controlled so as to obtain an EDOF effect according to a surgery mode, and an appropriately focused image can be displayed.

Note that, in the present embodiment, a configuration example using three pieces of imaging elements 52-1 to 52-3 has been described. However, for example, the present technology can be applied to a configuration using at least two pieces of imaging elements 52-1 and 52-2. In such a configuration, for example, three types of images, a Near image, a Mid image, and an EDOF image can be selectively output according to the surgery mode.

### Configuration Example of Computer

Next, the series of processes (control method) described above can be performed by hardware or software. In a case where a series of processing is performed by software, a program that constitutes the software is installed on a general-purpose computer, or the like.

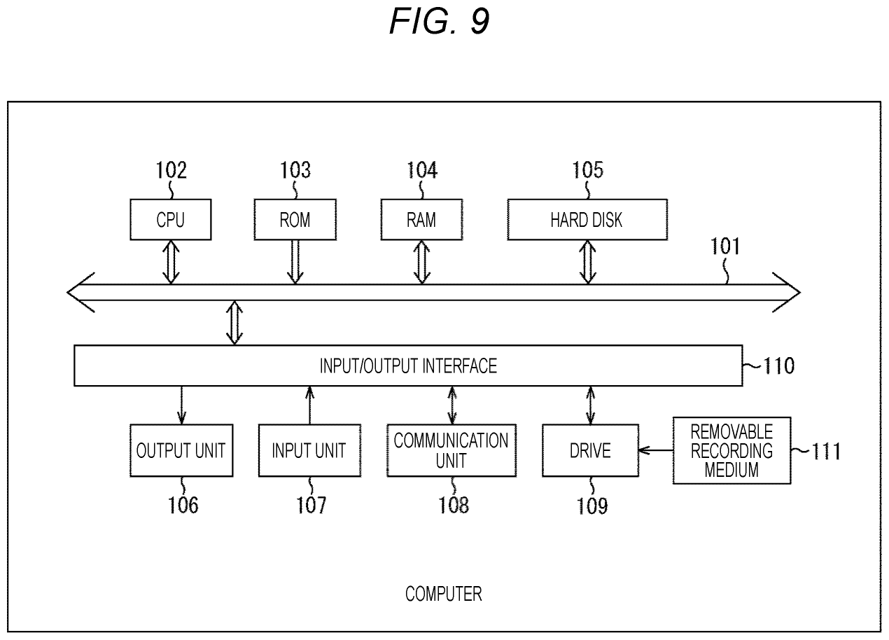

FIG. 9 is a block diagram illustrating a configuration example of an embodiment of a computer on which a program for executing the above-described series of processing is installed.

The program can be previously recorded on a hard disk 105 or ROM 103 as a recording medium incorporated in the computer.

Alternatively, the program can be stored (recorded) in a removable recording medium 111 driven by a drive 109. Such a removable recording medium 111 can be provided as so-called package software. Here, examples of the removable recording medium 111 include, for example, a flexible disk, a compact disc read only memory (CD-ROM), a magneto optical (MO) disk, a digital versatile disc (DVD), a magnetic disk, a semiconductor memory, and the like.

Note that in addition to installing the program on the computer from the removable recording medium 111 as described above, the program can be downloaded to the computer via a communication network or a broadcasting network and installed on the incorporated hard disk 105. That is, for example, the program can be wirelessly transferred from a download site to the computer via an artificial satellite for digital satellite broadcasting, or can be transferred by wire to the computer via a network such as a local area network (LAN) and the Internet.

The computer has a built-in central processing unit (CPU) 102, and an input/output interface 110 is connected to the CPU 102 via a bus 101.

When a command is inputted by a user operating an input unit 107 or the like via the input/output interface 110, in response to this, the CPU 102 executes a program stored in the read only memory (ROM) 103. Alternatively, the CPU 102 loads a program stored in the hard disk 105 into a random access memory (RAM) 104 and executes the program.

With this arrangement, the CPU 102 performs processing according to the above-described flowchart or processing performed according to the above configuration described with the block diagram. Then, as necessary, the CPU 102 outputs a processing result from an output unit 106, transmits the processing result from a communication unit 108, causes the hard disk 105 to record the processing result, or the like, via the input/output interface 110, for example.

Note that the input unit 107 includes a keyboard, a mouse, a microphone, or the like. Furthermore, the output unit 106 includes a liquid crystal display (LCD), a speaker, and the like.

Here, in this specification, the processing to be performed by the computer in accordance with the program is not necessarily performed in chronological order according to the sequences described in the flowcharts. That is, the processing to be performed by the computer in accordance with the program includes processing to be executed in parallel or independently of one another (parallel processing or object-based processing, for example).

Furthermore, the program may be processed by one computer (processor) or processed in a distributed manner by a plurality of computers. Moreover, the program may be transferred to a distant computer and executed.

Moreover, in the present description, a system means a set of a plurality of components (devices, modules (parts), and the like), and it does not matter whether or not all components are in the same housing. Therefore, a plurality of devices housed in separate housings and connected via a network, and one device in which a plurality of modules is housed in one housing are both systems.

Furthermore, for example, it is also possible to divide the configuration described as one device (or processing unit) into a plurality of devices (or processing units). Other way round, it is also possible to put the configurations described above as a plurality of devices (or processing units) together as one device (or processing unit). Furthermore, a configuration other than the above-described configurations may be added to the configuration of each device (or each processing unit). Moreover, when the configuration and operation of the entire system are substantially the same, part of the configuration of a certain device (or processing unit) may be included in the configuration of another device (or another processing unit).

Furthermore, for example, the present technology can be configured as cloud computing in which one function is shared and jointly processed by a plurality of devices via a network.

Furthermore, for example, the above-described program can be executed by an arbitrary device. In this case, it is sufficient that the device has a necessary function (functional block or the like) and is only required to obtain necessary information.

Furthermore, for example, respective steps described in the above-described flowcharts can be executed by one device or can be executed in a shared manner by a plurality of devices. Moreover, in a case where a plurality of processing is included in one step, the plurality of processing included in the one step can be executed by a single device or shared and executed by a plurality of devices. In other words, a plurality of pieces of processing included in one step may be executed as processes of a plurality of steps. Conversely, the processing described as a plurality of steps can be collectively executed as one step.

Note that, the program executed by the computer may be a program in which processing in steps describing the program is executed in time series in an order described in the present specification, or a program in which the processing may be executed in parallel, or individually at a necessary timing such as when a call is made. That is, unless there is a contradiction, the processing of each step may be executed in an order different from the order described above. Moreover, this processing in steps describing program may be executed in parallel with processing of another program, or may be executed in combination with processing of another program.

Note that a plurality of pieces of the present technology which has been described in the present description can each be implemented independently as a single unit as long as no contradiction occurs. Needless to say, a plurality of arbitrary pieces of the present technology can be used in combination. For example, part or all of the present technology described in any of the embodiments can be implemented in combination with part or all of the present technologies described in other embodiments. Furthermore, part or all of any of the above-described present technology can be implemented using together with another technology that is not described above.

### Examples of Configuration Combinations

Note that the present technology can also have the following configuration.

(1)

A medical imaging system including

a surgery mode setting unit that sets a surgery mode,

a region-of-interest setting unit that sets, on the basis of the surgery mode, a region of interest (ROI) image used for performing auto focus (AF) processing, from among two or more types of images captured by at least two pieces of imaging elements having different optical path lengths from one imaging lens, and, in the ROI image, sets a region of interest that is an area for which an evaluation value of contrast AF is obtained, and

a focus processing unit that obtains an evaluation value from a region of interest of the ROI image, and adjusts focus.

(2)

The medical imaging system according to (1),

in which a Near image focused on a near point, a Mid image focused on a middle point, and a Far image focused on a far point are used, the Near image, the Mid image, and the Far image being captured by three pieces of imaging elements having different optical path lengths from one imaging lens.

(3)

The medical imaging system according to (2), the medical imaging system further including a selection map generation unit that obtains contrast for each pixel of the Near image, Mid image, and Far image, selects, from among the Near image, the Mid image, and the Far image, an image from which highest contrast is obtained, and generates a selection map in which an identification number for identifying the selected image is associated with all pixels,

in which focus adjustment is repeatedly performed by the focus processing unit so that the identification number of the region of interest in the selection map matches an image set as the ROI image.

(4)

The medical imaging system according to (2) or (3), the medical imaging system further including an EDOF image generation unit that generates an extended depth of field (EDOF) image obtained by obtaining contrast for each pixel of the Near image, Mid image, and Far image that are captured with the region of interest of the ROI image being in focus by the focus processing unit, and selecting and combining pixels having highest contrast.

(5)

The medical imaging system according to any one of (2) to (4),

in which, in a case where a surgery mode in which a surgery of an anterior eye is performed is set, the region-of-interest setting unit sets the ROI image to the Near image, and sets the region of interest to a cornea appearing in the ROI image.

(6)

The medical imaging system according to any one of (2) to (5),

in which, in a case where a surgery mode in which a surgery of a fundus region is performed is set, the region-of-interest setting unit sets the ROI image to the Far image, and sets the region of interest to a fundus appearing in the ROI image.

(7)

The medical imaging system according to any one of (2) to (6),

in which, in a case where a surgery mode in which a surgery of a crystalline lens is performed is set, the region-of-interest setting unit sets the ROI image to the Mid image, and sets the region of interest to a crystalline lens appearing in the ROI image.

(8)

The medical imaging system according to any one of (2) to (7),

in which, in a case where a surgery mode in which is used as a laparoscope to perform a surgery is performed is set, the region-of-interest setting unit sets the ROI image to the Mid image, and sets the region of interest to the center of the ROI image.

(9)

A medical imaging device including

a surgery mode setting unit that sets a surgery mode,

a region-of-interest setting unit that sets, on the basis of the surgery mode, a region of interest (ROI) image used for performing auto focus (AF) processing, from among two or more types of images captured by at least two pieces of imaging elements having different optical path lengths from one imaging lens, and, in the ROI image, sets a region of interest that is an area for which an evaluation value of contrast AF is obtained, and

a focus processing unit that obtains an evaluation value from a region of interest of the ROI image, and adjusts focus.

(10)

A control method including, by a medical imaging system

setting a surgery mode,

setting, on the basis of the surgery mode, a region of interest (ROI) image used for performing auto focus (AF) processing, from among two or more types of images captured by at least two pieces of imaging elements having different optical path lengths from one imaging lens, and, in the ROI image, setting a region of interest that is an area for which an evaluation value of contrast AF is obtained, and

obtaining an evaluation value from a region of interest of the ROI image, and adjusting focus.

Note that the present embodiment is not limited to the above-described embodiment, and various modifications can be made without departing from the gist of the present disclosure. Furthermore, the effects described in the present specification are merely examples and are not limited, and other effects may be provided.

### REFERENCE SIGNS LIST

11 Medical imaging system
12 Endoscope
13 Energy treatment tool
14 Display device
15 Device unit
16 Forceps
21 Camera head
22 Lens barrel unit
31 Light source device
32 CCU
33 Recording device
34 Output device
41 Imaging module
42 Imaging lens
51 Optical splitting system
52-1 to 52-3 Imaging element
61 First prism
62 Second prism
63 Third prism
64 First dichroic mirror
65 Second dichroic mirror
71 Surgery mode setting unit
72 Region-of-interest setting unit
73 Imaging signal acquisition unit
74 EDOF image output unit
75 Color-coded image output unit
76 Focus processing unit
77 Selection map generation unit

1. A medical imaging system comprising:
a surgery mode setting unit that sets a surgery mode;
a region-of-interest setting unit that sets, on a basis of the surgery mode, a region of interest (ROI) image used for performing auto focus (AF) processing, from among two or more types of images captured by at least two pieces of imaging elements having different optical path lengths from one imaging lens, and, in the ROI image, sets a region of interest that is an area for which an evaluation value of contrast AF is obtained; and
a focus processing unit that obtains an evaluation value from a region of interest of the ROI image, and adjusts focus.

2. The medical imaging system according to claim 1,
wherein a Near image focused on a near point, a Mid image focused on a middle point, and a Far image focused on a far point are used, the Near image, the Mid image, and the Far image being captured by three pieces of imaging elements having different optical path lengths from one imaging lens.

3. The medical imaging system according to claim 2, the medical imaging system further comprising a selection map generation unit that obtains contrast for each pixel of the Near image, Mid image, and Far image, selects, from among the Near image, the Mid image, and the Far image, an image from which highest contrast is obtained, and generates a selection map in which an identification number for identifying the selected image is associated with all pixels,
wherein focus adjustment is repeatedly performed by the focus processing unit so that the identification number of the region of interest in the selection map matches an image set as the ROI image.

4. The medical imaging system according to claim 2, the medical imaging system further comprising an EDOF image generation unit that generates an extended depth of field (EDOF) image obtained by obtaining contrast for each pixel of the Near image, Mid image, and Far image that are captured with the region of interest of the ROI image being in focus by the focus processing unit, and selecting and combining pixels having highest contrast.

5. The medical imaging system according to claim 2,
wherein, in a case where a surgery mode in which a surgery of an anterior eye is performed is set, the region-of-interest setting unit sets the ROI image to the Near image, and sets the region of interest to a cornea appearing in the ROI image.

6. The medical imaging system according to claim 2,
wherein, in a case where a surgery mode in which a surgery of a fundus region is performed is set, the region-of-interest setting unit sets the ROI image to the Far image, and sets the region of interest to a fundus appearing in the ROI image.

7. The medical imaging system according to claim 2,
wherein, in a case where a surgery mode in which a surgery of a crystalline lens is performed is set, the region-of-interest setting unit sets the ROI image to the Mid image, and sets the region of interest to a crystalline lens appearing in the ROI image.

8. The medical imaging system according to claim 2,
wherein, in a case where a surgery mode in which is used as a laparoscope to perform a surgery is performed is set, the region-of-interest setting unit sets the ROI image to the Mid image, and sets the region of interest to a center of the ROI image.

9. A medical imaging device comprising:
a surgery mode setting unit that sets a surgery mode;
a region-of-interest setting unit that sets, on a basis of the surgery mode, a region of interest (ROI) image used for performing auto focus (AF) processing, from among two or more types of images captured by at least two pieces of imaging elements having different optical path lengths from one imaging lens, and, in the ROI image, sets a region of interest that is an area for which an evaluation value of contrast AF is obtained; and
a focus processing unit that obtains an evaluation value from a region of interest of the ROI image, and adjusts focus.

10. A control method comprising, by a medical imaging system:
setting a surgery mode;
setting, on a basis of the surgery mode, a region of interest (ROI) image used for performing auto focus (AF) processing, from among two or more types of images captured by at least two pieces of imaging elements having different optical path lengths from one imaging lens, and, in the ROI image, setting a region of interest that is an area for which an evaluation value of contrast AF is obtained; and
obtaining an evaluation value from a region of interest of the ROI image, and adjusting focus.
